(19) United States
(12) Patent Application Publication
(10) Pub. No.: US 2026/0077010 A1
(43) Pub. Date: Mar. 19, 2026

(54) COMPLEX WITH STROKE THERAPEUTIC CHARACTERISTIC, AND PREPARATION METHOD AND USE THEREOF

(71) Applicant: Beijing Technology and Business University, Beijing (CN)

(72) Inventors: Hongzhi Liu, Beijing (CN); Xuchun Zhu, Beijing (CN); Shanyue Guan, Beijing (CN); Bingyu Chen, Beijing (CN); Chao Yan, Beijing (CN); Tong Meng, Beijing (CN); Mengfan Li, Beijing (CN)

(73) Assignee: Beijing Technology and Business University, Beijing (CN)

(21) Appl. No.: 19/023,413

(22) Filed: Jan. 16, 2025

(30) Foreign Application Priority Data
Sep. 13, 2024 (CN) 2024112857813

(51) Int. Cl. A61K 38/01; A61K 31/05; A61P 9/10; C12N 1/16; C12N 9/50; C12P 21/06

(52) U.S. Cl. CPC A61K 38/01; A61K 31/05; A61P 9/10; C12N 1/16; C12N 9/63; C12P 21/06; C12Y 304/22002

(57) ABSTRACT

The present invention discloses a complex with a stroke therapeutic characteristic, and a preparation method and use thereof. The complex is a complex of a yeast peptide and hydroxytyrosol. The yeast peptide is a yeast peptide with a molecular weight less than 3 kDa produced by papain enzymolysis of yeast protein. The pH during enzymatic hydrolysis is 6.8-7.2, the enzymatic hydrolysis time is 3.8-6.2 h, the enzymatic hydrolysis temperature is 48-58° C., and the ratio of the papain to the yeast protein in the enzymatic hydrolysis system is 8,800-9,200 U/g. The complex is used for manufacture of a drug for treating ischemic stroke. The present invention offers an efficient, safe, and cost-effective complex with a stroke therapeutic characteristic, featuring broad application prospects, and significant technical advantages, and the complex has a simple preparation method and low production cost.

### TECHNICAL FIELD

The present invention relates to the field of biomedical technology, and in particular to a complex with a stroke therapeutic characteristic, and a preparation method and use thereof.

### BACKGROUND OF THE INVENTION

Stroke is an acute cerebrovascular disease with features of high morbidity, high disability and high mortality, which has brought a heavy burden to society and families. The changes in the brain microenvironment caused by blood reperfusion after ischemic stroke are the main obstacle to the recovery of neurological function, and are also a key factor in the recurrence of stroke after thrombolytic therapy. During blood perfusion restoration, brain cells exacerbate injury caused by ischaemia and hypoxia or trigger complex reactions, resulting in ischaemia-reperfusion injury (IRI), neuronal necrosis, and impaired neurological function.

Currently, the treatment of stroke mainly relies on means such as thrombolytic therapy, antiplatelet therapy and neuroprotective therapy, but these methods have certain limitations. For example, the thrombolytic therapy is limited by problems such as a narrow treatment time window and a high risk of bleeding; the antiplatelet therapy may cause side effects such as bleeding and gastrointestinal discomfort; and the existing neuroprotective drugs have limited efficacy and are difficult to meet clinical needs.

In recent years, with the continuous development of biotechnology, scientific researchers have begun to explore utilization of new biomolecules such as polypeptides and nanozymes as potential drugs for the treatment of stroke. For example, a team led by researcher Lai Ren from the Kunming Institute of Zoology collaborated with Professor Ni Heyu et al from the University of Toronto to identify an active polypeptide with significant therapeutic effects from forest leeches (Haemadipsa sylvestris). This polypeptide can specifically inhibit the activity of kallikrein and FXIIa, demonstrates good therapeutic effects on both transient and permanent ischemic strokes, and has strong biological safety with anti-thrombotic effects and no bleeding risk, making it a promising candidate for drug development.

While polypeptides have potential in ischemic stroke treatment, they face some challenges, e.g. potential bioactivity loss during digestion and the generation of toxic or allergenic residues. Therefore, it is necessary to develop new, efficient, safe, and relatively low-cost polypeptides with therapeutic effects on stroke to meet clinical needs.

### SUMMARY OF THE INVENTION

Therefore, the technical problem to be addressed by the present invention is to provide an efficient, safe, and economical complex with a stroke treatment characteristic, and a preparation method and use thereof. The preparation method is simple, cost-effective, and has good therapeutic effects and biosafety.

In order to solve the above technical problems, the present invention provides the following technical solutions:

A complex with a stroke therapeutic characteristic, comprising a yeast peptide and hydroxytyrosol, the yeast peptide is a yeast peptide with a molecular weight less than 3 kDa produced by papain enzymolysis of a yeast protein; the pH during enzymatic hydrolysis is 6.8-7.2, the enzymatic hydrolysis time is 3.8-6.2 h, the enzymatic hydrolysis temperature is 48-58° C., and the amount/mass ratio of the papain to the yeast protein in the enzymatic hydrolysis system is 8,800-9,200 U/g. Experiments have demonstrated that yeast peptides with a molecular weight of less than 3 kDa, prepared using papain under the aforementioned enzymatic hydrolysis conditions, have a certain alleviating effect on stroke in mice. Furthermore, the complex formed by this yeast peptide and hydroxytyrosol has also shown, in mouse experiments, an effect in alleviating stroke and reversing oxidative damage during ischemia-reperfusion.

The complex with a stroke therapeutic characteristic, further comprising lecithin and/or a phospholipid membrane prepared using lecithin, the phospholipid membrane is prepared by dissolving the lecithin in chloroform and then evaporating the chloroform. Introducing lecithin or a phospholipid membrane into the complex can promote the binding of the yeast peptide and the hydroxytyrosol.

The complex with a stroke therapeutic characteristic, when the lecithin is added into the complex: the molar ratio of the yeast peptide, the hydroxytyrosol and the lecithin is (6.5-38.9):(6.5-38.9):(0.1-0.5); and when the phospholipid membrane is added into the complex, the amount of the phospholipid membrane is calculated according to the amount of the lecithin used for preparing the phospholipid membrane: the molar ratio of the yeast peptide, the hydroxytyrosol and the phospholipid membrane is (6.5-38.9):(6.5-38.9):(0.1-0.5).

A method for preparing a complex with a stroke therapeutic characteristic, comprising the following steps:
(1) preparing yeast protein powder prepared from a yeast into a yeast protein powder dispersion;
(2) placing and shaking the yeast protein powder dispersion under an enzymatic hydrolysis reaction temperature;
(3) adding papain to the yeast protein powder dispersion for enzymatic hydrolysis, so as to obtaining an enzymatic hydrolyzate dispersion after the enzymatic hydrolysis is completed;
(4) heating the enzymatic hydrolyzate dispersion to a protease inactivation temperature and maintaining at this temperature, and then cooling the enzymatic hydrolyzate dispersion and adjusting the pH of the enzymatic hydrolyzate dispersion;
(5) centrifuging the enzymatic hydrolyzate dispersion and retaining the supernatant, and sequentially ultrafiltering and freeze-drying the supernatant to obtain a yeast peptide having a molecular weight less than 3 kDa;
(6) dispersing the yeast peptide and the hydroxytyrosol in a phosphate buffer to form a composite system, and stirring to obtain a yeast peptide-hydroxytyrosol complex dispersion;
(7) purifying and freeze-drying the yeast peptide-hydroxytyrosol complex dispersion to obtain a yeast peptide-hydroxytyrosol complex, wherein the yeast peptide-hydroxytyrosol complex is the complex with a stroke therapeutic characteristic as above.

The method for preparing a complex with a stroke therapeutic characteristic, in step (1), a phosphate buffer is used for formulating a yeast protein powder dispersion, the pH of the phosphate buffer is equal to the pH during enzymatic hydrolysis; the content of the yeast protein in the yeast protein powder is greater than or equal to 80 wt %, and the concentration of the yeast protein powder in the yeast protein powder dispersion is 48-50 g/L.

The method for preparing a complex with a stroke therapeutic characteristic, in step (2), the shaking time is 10-15 min; in step (4), the protease inactivation temperature is 95-100° C., and the heat preservation time is 10-15 minutes; after the enzymatic hydrolysate is naturally cooled, the pH of the enzymatic hydrolysate is adjusted to 7.0.

The method for preparing a complex with a stroke therapeutic characteristic, in step (5), during centrifugation of the enzymatic hydrolysate, the centrifugation temperature is 4-8° C., the rotation speed during centrifugation is 10,000-12,000 rpm, and the centrifugation time is 10-20 min; in step (6), the molar ratio of the yeast peptide to the hydroxytyrosol in the composite system is 1:1, and the concentration of the hydroxytyrosol in the composite system is 1-6 mg/mL; after the composite system is prepared, lecithin and/or a phospholipid membrane made of lecithin is added into the composite system; the molar amount of the phospholipid membrane in the composite system is calculated according to the molar amount of the lecithin, and the molar concentration of the lecithin in the composite system is 0.1-0.5 mmol/L.

The method for preparing a complex with a stroke therapeutic characteristic, in step (6), the composite system is subjected to cyclic heating during the stirring process; the cyclic heating method is to heat the composite system to 48-52° C. at a heating rate of 3-7° C./min, maintaining at this temperature for 20-30 min, and then naturally cooling to room temperature until the stirring is completed; wherein the stirring time is 20-26 h.

The method for preparing a complex with stroke therapeutic characteristic, in step (1), a yeast protein powder dispersion is prepared using a phosphate buffer, the pH of the phosphate buffer is 7.0; the content of the yeast protein in the yeast protein powder is greater than or equal to 80 wt %, and the concentration of the yeast protein powder in the yeast protein powder dispersion is 50 g/L; in step (2), the shaking time is 10 min; in step (3), the enzymatic hydrolysis time is 6 h, the enzymatic hydrolysis temperature is 55° C., the pH during enzymatic hydrolysis is 7, and the ratio of the papain to the yeast protein in the enzymatic hydrolysis system is 9,000 U/g; in step (4), the protease inactivation temperature is 100° C., and the heat preservation time is 15 min; after the enzymatic hydrolyzate dispersion is naturally cooled, the pH of the enzymatic hydrolyzate dispersion is adjusted to 7.0; in step (5), during centrifugation of the enzymatic hydrolyzate dispersion, the centrifugation temperature is 4° C., the centrifugation speed is 10,000 rpm, and the centrifugation time is 15 min; in step (6), the molar ratio of the yeast peptide to the hydroxytyrosol in the composite system is 1:1, and the concentration of the hydroxytyrosol in the composite system is 5 mg/mL; after the composite system is prepared, lecithin and/or a phospholipid membrane made of lecithin is added into the composite system; the amount of the phospholipid membrane in the composite system is calculated according to the molar concentration of the lecithin, and the molar concentration of the lecithin in the composite system is 0.1 mmol/L; during the stirring process, the composite system is subjected to cyclic heating; the method of the cyclic heating is to heat the composite system to 50° C. at a heating rate of 5° C./min, maintaining at this temperature for 30 min, and then naturally cooling to room temperature until the stirring ends; and the stirring time is 24 h. When the yeast peptide and the hydroxytyrosol are compounded at a molar ratio of 1:1, the contact and binding between the yeast peptide and the hydroxytyrosol are more efficient. Cyclic heating can promote the full contact and binding of the yeast peptide and the hydroxytyrosol and improve the formation efficiency of the complex. The control of temperature helps to improve the stability of the complex, avoid the degradation or inactivation of components caused by high temperature, and thus enhance the efficacy of the complex.

The use of the compound with a stroke treatment characteristic in manufacturing a drug for treating ischemic stroke, is disclosed.

The technical solution of the present invention achieves the following beneficial technical effects:

1. The present invention provides a complex with a stroke therapeutic characteristic. This complex is prepared by compounding the yeast peptide and the hydroxytyrosol and demonstrates significant therapeutic characteristics for ischemic stroke. The results of animal experiments show that the cerebral infarction area in mice injected with the yeast peptide-hydroxytyrosol complex is significantly reduced, and their neurological scores are also notably improved. The complex can decrease the content of reactive oxygen species in the brain tissues of the mice, enhance superoxide dismutase activity, increase the content of the antioxidant glutathione, and reverse oxidative damage during ischemia-reperfusion, all without obvious toxic side effects. This complex is expected to become a new drug for stroke treatment, providing a safer and more effective therapeutic means for clinical use.
2. The complex of the present invention is prepared by enzymatic hydrolysis of the yeast protein using papain, which offers a simple process with readily available raw materials and low production costs. Compared to some polypeptide drugs that need to be extracted from special organisms, the complex of the present invention is more economical and feasible.

### BRIEF DESCRIPTION OF THE DRAWINGS

FIG. 1 TCC staining results of the brains of mice in a sham-operation group and MCAO mice injected with a PBS buffer, a yeast peptide, and a yeast peptide-hydroxytyrosol complex in examples of the present invention (the abscissa in the figure: “Sham” represents a sham operation group, “PBS” represents a group of MCAO mice injected with a PBS buffer, “yeast peptide” represents a group of MCAO mice injected with a “Mu-3 sample” yeast peptide dispersion, and “LP@complex” represents a group of MCAO mice injected with “Mu-3 complex”, a yeast peptide-hydroxytyrosol complex dispersion);

FIG. 2 Statistical results of the cerebral infarction areas of the mice in the sham-operation group and the MCAO mice injected with the PBS buffer, the yeast peptide, and the yeast peptide-hydroxytyrosol complex in examples of the present invention (the abscissa in the figure: “Sham” represents a sham operation group, “PBS” represents a group of MCAO mice injected with a PBS buffer, “yeast peptide” represents a group of MCAO mice injected with a “Mu-3 sample” yeast peptide dispersion, and “LP@complex” represents a group of MCAO mice injected with “Mu-3 complex”, a yeast peptide-hydroxytyrosol complex dispersion);

FIG. 3 Neurological score results of the mice in the sham-operation group and the MCAO mice injected with the PBS buffer, the yeast peptide, and the yeast peptide-hydroxytyrosol complex in examples of the present invention (the abscissa in the figure: “Sham” represents a sham operation group, “PBS” represents a group of MCAO mice injected with a PBS buffer, “yeast peptide” represents a group of MCAO mice injected with a “Mu-3 sample” yeast peptide dispersion, and “LP@complex” represents a group of MCAO mice injected with “Mu-3 complex”, a yeast peptide-hydroxytyrosol complex dispersion);

FIG. 4 Determination results of ROS content in brain tissues of the mice in the sham-operation group and mice in a tMCAO model injected with the PBS buffer, the yeast peptide, and the yeast peptide-hydroxytyrosol complex in examples of the present invention;

FIG. 5 SOD activity determination results of brain tissues from the mice in the sham-operation group and the mice in the tMCAO model injected with the PBS buffer, the yeast peptide, and the yeast peptide-hydroxytyrosol complex in examples of the present invention;

FIG. 6 Determination results of GSH content in the brain tissues of the mice in the sham-operation group and the mice in the tMCAO model injected with the PBS buffer, the yeast peptide, and the yeast peptide-hydroxytyrosol complex in examples of the present invention;

FIG. 7 Determination results of MDA content in the brain tissues of the mice in the sham-operation group and the mice in the tMCAO model injected with the PBS buffer, the yeast peptide, and the yeast peptide-hydroxytyrosol complex in examples of the present invention;

FIG. 8 Number of yeast peptides less than 1 kDa and between ≥1 kDa and <3 kDa in the papain hydrolysate of the yeast proteins prepared in examples of the present invention; in the figure, “<3 kDa” represents yeast peptides with a molecular weight of ≥1 kDa and <3 kDa;

FIG. 9 Size distribution of peptides in the fractions less than 1 kDa and in the fractions between ≥1 kDa and <3 kDa of the papain hydrolysate of the yeast proteins prepared in examples of the present invention; where the horizontal axis represents the number of amino acids contained in the peptides, and the vertical axis represents the percentage of the number of corresponding peptides to the total number of peptides;

FIG. 10 Percentage of various amino acids in the fractions less than 1 kDa and in the fractions between ≥1 kDa and <3 kDa of the papain hydrolysate of yeast protein prepared in examples of the present invention;

FIG. 11 Number of various amino acids in the fractions less than 1 kDa and in the fractions between ≥1 kDa and <3 kDa of the papain hydrolysate of yeast protein prepared in examples of the present invention.

### DETAILED DESCRIPTION OF THE EMBODIMENTS

### Example

In this example, a method for preparing a complex with a stroke therapeutic characteristic was specifically as follows:

(1) Angel AnPro80 yeast protein powder prepared from yeasts, was selected. This yeast protein powder had a nominal yeast protein content of 80 wt %. 50 g of the yeast protein powder was weighed and formulated into a yeast protein powder dispersion. The specific method was adding 50 g of the yeast protein powder into a 1 L phosphate buffer solution (PBS buffer) with a pH of 7.0, and thoroughly shaken to obtain a yeast protein powder dispersion with a concentration of 50 g/L.
(2) The yeast protein powder dispersion was placed into a thermostatic oscillator in a water bath of 55° C. and shaken for 10 min. After this step, the temperature of the dispersion was stabilized at 55° C., so as to ensure the subsequent enzymatic hydrolysis to occur under appropriate temperature conditions from the beginning, thereby improving both the efficiency and effectiveness of the reaction.
(3) papain (8.0×105 U/mg) was added into the yeast protein powder dispersion to enzymatically hydrolyze the yeast protein. 9,000 U of papain was added per gram of the yeast protein. Based on the yeast protein content of 80 wt % in the yeast protein powder in this example, the concentration of the yeast protein in the dispersion was 40 g/L. That was, 360,000 U of papain per liter of the yeast protein powder dispersion was added. The hydrolysis was performed for 6 hours at a temperature of 55° C. [In some other examples, the enzymatic hydrolysis temperature could also be set to 50° C. with an enzymatic hydrolysis duration of 4 h. Correspondingly, the water bath temperature in step (2) was set to 50° C.; however, under these enzymatic hydrolysis conditions, the content of yeast peptides with a molecular weight less than 3 kDa obtained through enzymatic hydrolysis was relatively low.] After end of the enzymatic hydrolysis, an enzymatic hydrolyzate dispersion was obtained.
(4) The enzymatic hydrolyzate dispersion was heated to 100° C. (protease inactivation temperature) and maintained at this temperature for 15 min to inactivate the protease (In other examples, the holding time could also be set at 10 min). The enzymatic hydrolyzate dispersion was allowed to be cooled naturally to room temperature, and then adjusted to a pH of 7.0 using 1 mol/L of NaOH or 1 mol/L of HCl.
(5) The pH-adjusted enzymatic hydrolysate dispersion was centrifuged using a low-temperature, high-speed centrifuge at a centrifuging temperature of 4° C., a rotation speed of 10,000 rpm, for a centrifuging time of 15 min. The supernatant was retained after centrifugation.

A portion of the supernatant was taken and freeze-dried to obtain a yeast peptide. Subsequently, the yeast peptide was formulated into a 1% (w/v) dispersion of the yeast peptide. This yeast peptide dispersion was injected into a chromatograph through a 0.45 μm microporous filter for the determination of the molecular weight range of the yeast peptide. The chromatographic column was a TSK gel 2000 SWXL 300 mm×7.8 mm at a temperature of 30° C. with a flow rate of 0.50 mL/min and a wavelength of 220 nm. The molecular weight calibration standards were: cytochrome C (125.00 Da), bacillus subtilisin (1,450 Da), and acetyl-acetyl-tyrosine-arginine (451 Da). The determination results indicated that the yeast peptide prepared by the aforementioned method had a molecular weight of less than 5 kDa.

After the molecular weight distribution of the yeast peptide in the supernatant was determined, ultrafiltration was employed to separate yeast peptides of different molecular weights from the supernatant. To prevent large particles in the supernatant from clogging the ultrafiltration membrane, before ultrafiltration, the supernatant was sequentially passed through 0.45 μm and 0.22 μm microporous membranes to further remove the large particles. Subsequently, the supernatant, now devoid of the large particles, was ultrafiltered using 5 kDa, 3 kDa, and 1 kDa ultrafiltration membranes, respectively to separate enzymes-hydrolyzed products (yeast peptides) with molecular weights of less than 1 kDa, greater than or equal to 1 kDa and less than 3 kDa, and greater than or equal to 3 kDa and less than 5 kDa. These fractions were respectively freeze-dried to obtain yeast peptides of different molecular weights.

The interruption of cerebral blood flow during ischemic stroke caused local cerebral tissue ischemia and hypoxia, while the restoration of cerebral blood flow further caused cerebral ischemia-reperfusion injury (CIRI). It was well known that, micro vessels formed by compensatory angiogenesis or stimulated by exogenous factors (e.g., vascular endothelial growth factors (VEGFs)) after stroke are structurally abnormal, showing higher permeability and rupture susceptibility. This often led to reperfusion injury, particularly through the production of reactive oxygen species (ROSs), exacerbating vascular inflammation and dysfunction. Therefore, inhibiting oxidative stress was particularly important for alleviating neurological function damage in stroke.

It had been confirmed through extensive literature that antioxidant peptides generally contained more than three amino acid residues and usually consisted of three to six amino acids with molecular weights below 1 kDa. The molecular weight of the antioxidant peptides ranged from 400 to 650 Da. Some researchers had attempted to demonstrate and explain the relationship between molecular weight and antioxidant activity. For example, among the bioactive peptides extracted from alkaline protease-hydrolyzed residues from olive oil production, the antioxidant capacity of short chain peptides was significantly higher than that of high molecular weight peptides. Furthermore, peptide fractions with the molecular weight of less than 3 kDa exhibited the highest antioxidant activity in separation of zebra bean protein hydrolysates using ultrafiltration membranes with molecular weight cut-offs of 100, 50, 30, 10 and 3 kDa. Peptides with the molecular weight below 1 kDa exhibited the best initial and sustained antioxidant activity in ABTS+ radical scavenging, hydroxyl radical scavenging and ORAC value tests. In addition, in alkaline protease hydrolysates of egg white proteins, peptides with molecular weights less than 1 kDa had the strongest antioxidant capacity compared to other ultrafiltration fractions. In a linoleic acid model system of BNH-P7, the peptides with smaller molecular weights had higher activity against lipid peroxidation, as longer peptide sequences (282 peptides) led to a dilution effect on peptides exhibiting hydroxyl radical scavenging activity. Therefore, an appropriate low molecular weight significantly affected the antioxidant activity of the peptides.

The LC-MS/MS peptide sequence analysis was performed on the yeast peptides of less than 1 kDa and the yeast peptides of greater than or equal to 1 kDa and less than 3 kDa obtained by separation via ultrafiltration. A HPLC system (Easy nLC 1200, Thermo Fisher Scientific, Massachusetts, USA) was equipped with a C18 chromatographic column (3 μm, 100 Å, 75 μm×15 cm) was used during analysis, with phase A being 0.1% formic acid and phase B being 0.1% formic acid and 80% acetonitrile/water, at a flow rate of 0.3 mL/min. The elution process was: 95% phase A and 5% phase B over 0 to 4 minutes, 90% phase A and 10% phase B over 4 to 40 minutes, 72% phase A and 28% phase B over 40 to 47 minutes, 62% phase A and 38% phase B over 47 to 48 min, and 0% phase A and 100% phase B over 48 to 60 min. Analytical conditions of mass spectrometry (Orbitrap Fusion Lumos, Thermo Fisher Scientific, Massachusetts, USA) were set as follows: a spray voltage of 2.0 kV, a capillary temperature of 320° C., and an radio frequency (RF) lens voltage of 40 V. Resolution settings were 120,000@m/z 200 for primary mass spectrometry and 30,000@m/z 200 for secondary mass spectrometry. The precursor ion scan range was set to m/z 350-1,550. The production scan range started from m/z 110. The MS1 automatic gain control (AGC) was set to 4e5, and the ion injection time was 50 ms. The MS2 AGC was set to 1e5, and the ion injection time was set to 50 ms. The ion screening window width was 1.6 m/z, high-energy collision dissociation (HCD) was adopted as the mode of ion cleavage, the collision energy was set to NCE 32, the top 20 strongest ions were selected for fragmentation in data-dependent MS/MS, and the dynamic exclusion time was set to 60 s.

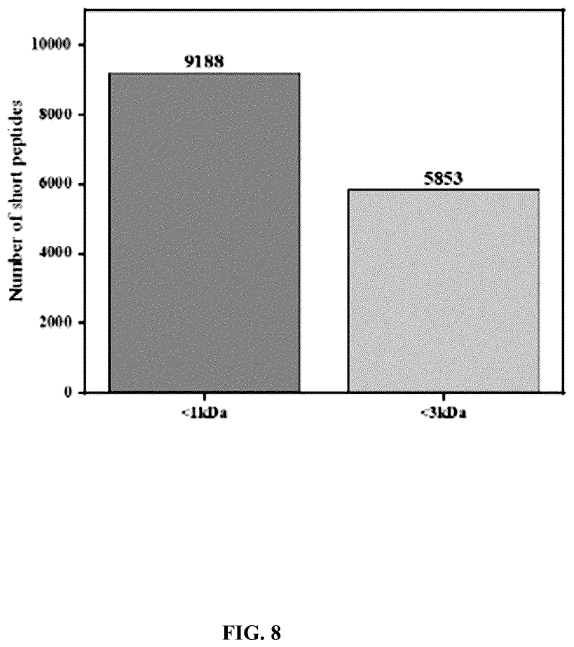

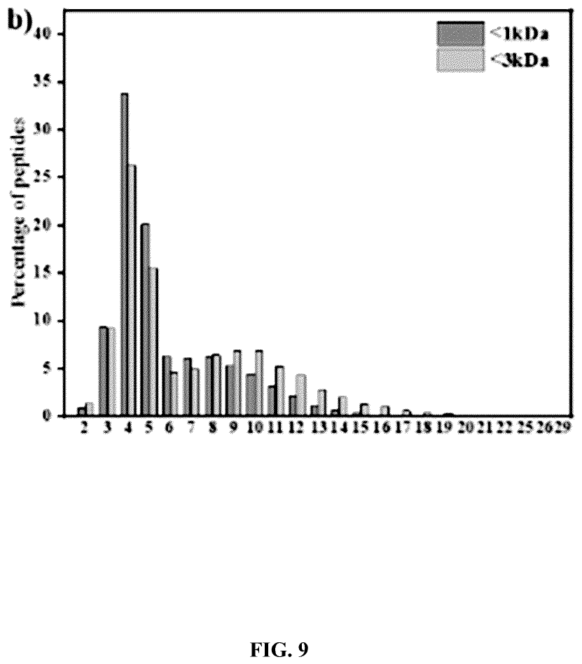

The yeast peptides with molecular weights less than 1 kDa were labeled as Mu-1, and the yeast peptides with molecular weights greater than or equal to 1 kDa and less than 3 kDa were labeled as Mu-3. The analysis results showed that 9,188 peptide segments were identified in the Mu-1 sample (FIG. 8), while 5,853 peptide segments were identified in the Mu-3 sample (FIG. 8). The content of tetrapeptides and pentapeptides in Mu-1 was significantly higher than that in Mu-3 (FIG. 9), and there were more peptides with molecular weights of less than 1 kDa in Mu-1, so the sample Mu-1 had stronger antioxidant activity.

According to the literature, an amino acid sequence is one of the key determinants of antioxidant characteristics. In particular, histidine (His) was considered as one of the major amino acids in antioxidant peptides. The antioxidant activity of histidine was mainly derived from its side chain imidazole groups. Previous studies had shown that the phenolic hydroxyl group of tyrosine (Tyr) and the indole group of tryptophan (Trp) could donate hydrogen, which in turn enhanced the antioxidant activity of the peptides containing these amino acids. Studies had shown that tryptophan (Trp), tyrosine (Tyr) and phenylalanine (Phe) could constitute peptides with high antioxidant capacity. Secondly, the hydrophobicity of amino acids also play an important role in the antioxidant activity. Hydrophobic amino acids could increase the solubility of peptides in lipid foods, thus improving their antioxidant properties. Moreover, the molecular weight was also one of factors affecting the antioxidant characteristic, and antioxidant peptides with smaller molecular weights not only had higher antioxidant activity, but also had better absorption in the body.

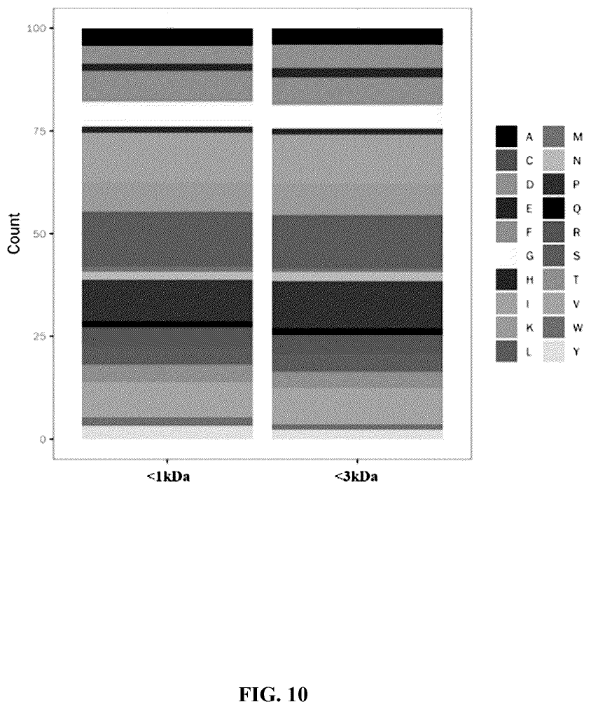

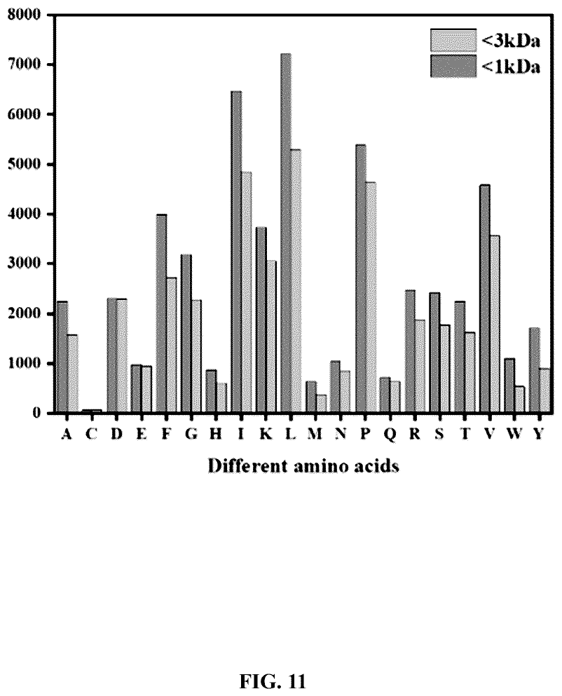

In this example, the yeast peptides obtained by hydrolyzing the yeast protein with papain were sequenced to detect the amino acid composition of each polypeptide. The results showed that among the yeast peptides obtained by papain hydrolysis of the yeast protein, the content of histidine and hydrophobic amino acids in the yeast peptide less than 1 kDa was higher than that in the yeast peptide greater than or equal to 1 kDa and less than 3 kDa (FIGS. 10 and 11), indicating that the Mu-1 sample had stronger antioxidant potential. The number of short peptides in the Mu-1 sample was significantly higher than that in the Mu-3 sample, so it was more likely that the yeast peptides with antioxidant characteristics appeared in the Mu-1 sample. In this example, the yeast peptide-hydroxytyrosol complexes was prepared from the yeast peptide with a molecular weight less than 1 kDa (i.e. the Mu-1 sample), and the yeast peptide with a molecular weight greater than or equal to 1 kDa and less than 3 kDa (i.e. the Mu-3 sample).

(6) The use amounts of the yeast peptide and the hydroxytyrosol were calculated, and they were mixed at a molar ratio of 1:1 (the molar amount of the yeast peptide being calculated based on the average molecular weight of the yeast peptide). The yeast peptide and the hydroxytyrosol were added into a PBS buffer to form a composite system. The concentration of the hydroxytyrosol in the composite system was 5 mg/mL, and the concentration of the yeast peptide in the composite system was 36.8 mg/mL. In other examples, the concentration of the hydroxytyrosol in the composite system might also be other values in a range from 1 to 6 mg/mL.

In order to enhance the binding of the yeast peptide and the hydroxytyrosol, lecithin or a phospholipid membrane made from lecithin was added to the complex at a concentration of 0.1 mmol/L (it could also be formulated at the concentration of the lecithin or the phospholipid membrane of any value between 0.1-0.5 mmol/L, with the amount of the phospholipid membrane calculated based on the molar amount of the lecithin used for preparing the phospholipid membrane). The phospholipid membrane was prepared by dissolving the lecithin in chloroform, followed by evaporation of the chloroform. The relatively hydrophilic head of the lecithin could bind with the alcohol hydroxyl group in the hydroxytyrosol and the hydrophilic groups in the yeast peptide, while the hydrophobic tail of the lecithin could bind with each other. Additionally, some hydrophobic groups in the yeast peptide can bind with the hydrophobic tail of the lecithin (without the addition of phospholipids, these hydrophobic groups cannot bind with the hydroxytyrosol). Therefore, the addition of the lecithin or the phospholipid membrane could facilitate the binding of e hydroxytyrosol and the yeast peptide, forming a stable complex.

After addition of the lecithin, the composite system was stirred while undergoing cyclic heating. The cyclic heating referred to heating the composite system to 50° C. at a rate of 5° C./min, maintaining at this temperature for 30 min, and then allowing it to cool naturally to room temperature. This heating and cooling cycle was repeated, and the system was continuously stirred for 24 h. Once stirring was completed, a yeast peptide-hydroxytyrosol complex dispersion was obtained.

In some other examples, the heating rate of the cyclic heating ranged from 3-7° C./min, with the temperature after heating ranging from 48-52° C., and the stirring time ranging from 20-26 h.

(7) The yeast peptide-hydroxytyrosol complex dispersion was purified. The aforementioned yeast peptide-hydroxytyrosol complex dispersion was transferred into a centrifuge tube equipped with a ultrafiltration membrane with a molecular cut off of 1 kDa, and then centrifuged. The operation conditions for the centrifugation were 4° C., a rotation speed of 10,000 rpm, and a centrifugation time of 15 minutes. When the yeast peptide-hydroxytyrosol complex was prepared by using the “Mu-3 sample” yeast peptide: after completion of the centrifugation, the filtrate that passed through the ultrafiltration membrane was carefully discarded and the contents above the membrane were retained. When the yeast peptide-hydroxytyrosol complex was prepared by using the “Mu-1 sample” yeast peptide, after completion of the centrifugation, the filtrate remained on the ultrafiltration membrane was carefully discarded and the filtrate under the ultrafiltration membrane was retained. To further remove any unbound yeast peptide and hydroxytyrosol, an appropriate amount of ultrapure water was added above the ultrafiltration membrane, mixed thoroughly, and then centrifuged again. The washing and centrifugation steps were repeated for a total of two times, each time centrifuging at 4° C. under the condition of 10,000 rpm for 15 minutes.

The yeast peptide-hydroxytyrosol complex prepared using the Mu-1 sample was labeled as a “Mu-1 complex,” and the yeast peptide-hydroxytyrosol complex prepared using the Mu-3 sample was labeled as a “Mu-3 complex”. To evaluate the therapeutic effects of the two prepared yeast peptide-hydroxytyrosol complexes on ischemic stroke, animal experiments were conducted. First, an ischemic stroke mice model (MCAO mice model) was established through surgery. The specific steps involved in establishing the model were as follows:

1. C57BL/6J mice were anesthetized by intraperitoneal injection of sodium pentobarbital (50 mg/kg) to ensure that the mice were completely unconscious.
2. An incision of 1 cm long was cut along the midline of the neck of the mouse. the skin and muscle were gently separated by using forceps and scissors to expose the right common carotid artery, internal carotid artery, and external carotid artery.
3. The common carotid artery, internal carotid artery, and external carotid artery were gently isolated and clamped by using hemostatic forceps to prevent blood flow. A small incision was made at the end of the external carotid artery (near a branching point) (this incision was the stump of the external carotid artery). A 30 mm long 6-0 suture was inserted into the internal carotid artery through the stump of the external carotid artery and slowly advanced to block the middle cerebral artery.

During the surgery, the body temperature of the mice was maintained between 37.0-37.5° C. by using a heating pad.

In addition, a set of sham-operation groups was additionally set. In the sham-operation group, no suture was inserted into the artery, and the rest of the steps were the same as those for constructing the aforementioned ischemic stroke mice model.

The MCAO (middle cerebral artery occlusion) mice were randomly divided into 3 groups, with 6 mice in each group. After 60 min of MCAO, the 3 groups of MCAO mice were subjected to tail intravenous injection of 200 μL of a PBS buffer, a yeast peptide dispersion (prepared with a PBS buffer, at a concentration of 0.01 M), and a yeast peptide-hydroxytyrosol complex dispersion (formulated with a PBS buffer, at a concentration of 0.01 M), respectively. Meanwhile, the mice in the sham-operation group were injected with an equal volume of the PBS buffer.

1.5 h after completion of the injection, the sutures in the blood vessels were removed to simulate clinical interventional thrombectomy and restore blood flow in the middle cerebral artery.

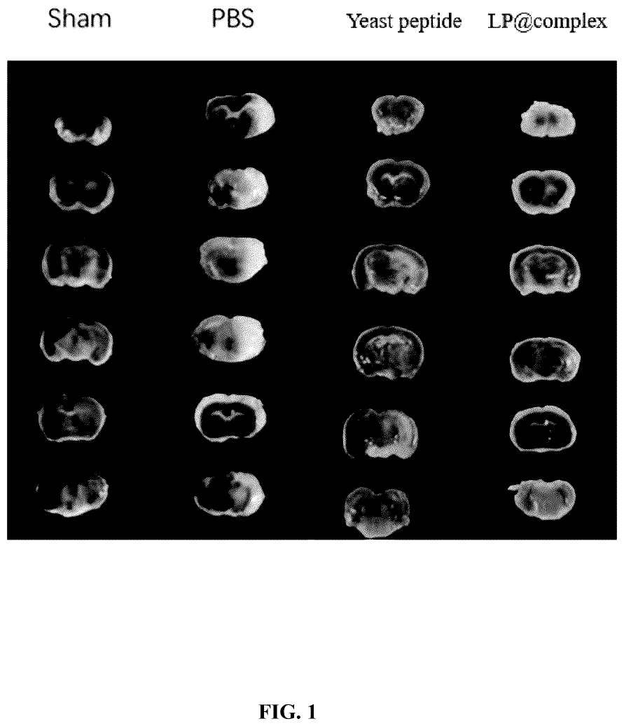

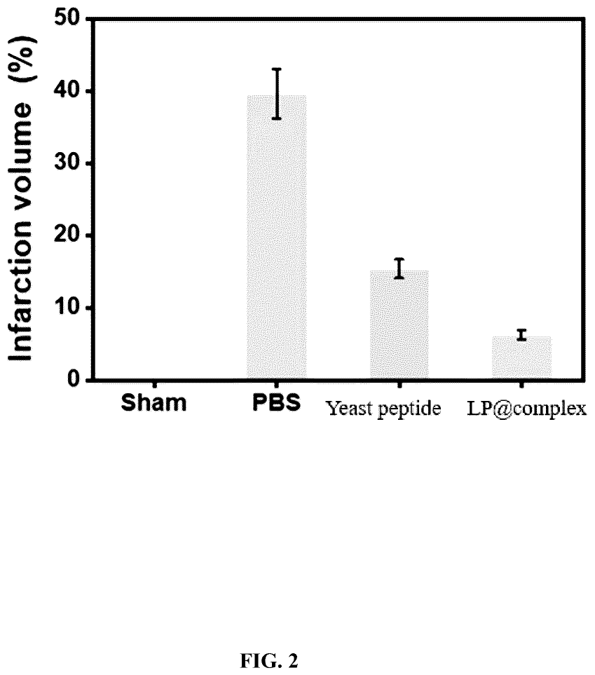

The mice were then evaluated with neurological scoring, after which they were sacrificed, and brain tissues were collected for TTC staining. The staining results were shown in FIG. 1, where the white areas represented a cerebral infarction region. The area of the cerebral infarction region was measured, and the proportion of the infarct area was calculated. The results were shown in FIG. 2. It could be seen from FIG. 2 that, the infarct area of the mice in the PBS group (the control group) was about 40% (i.e., the area of the infarction region accounting for 40% of the surface area of the brain), the infarct area of the mice in the yeast peptide group was about 15%, and the infarct area of mice in the yeast peptide-hydroxytyrosol complex group was about 5%. As seen from this experiment result, the injection of the yeast peptide-hydroxytyrosol complex dispersion had a significant therapeutic effect on the MCAO mice, could reduce the cerebral infarction area of the MCAO mice by about 87.5% (even compared to use of hydroxytyrosol alone, the infarction area was reduced by 58.3%).

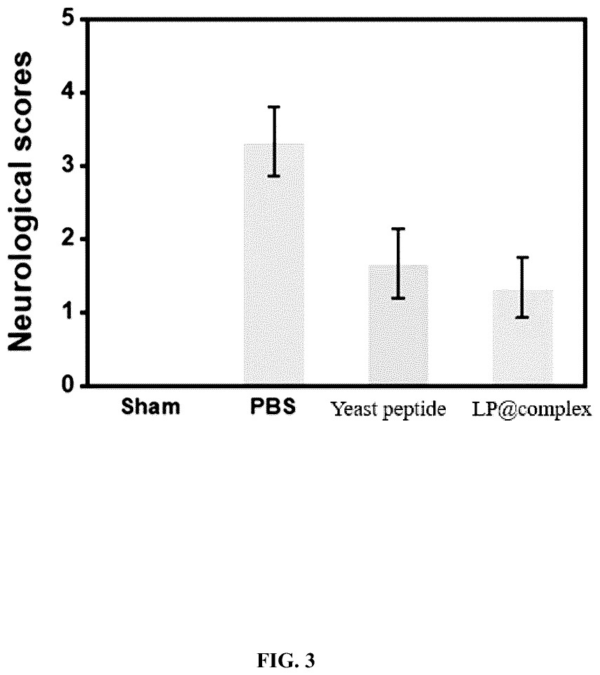

FIG. 3 showed the neurological score evaluation results for each group of mice. Neurological scoring was performed to assess the neurological function of the mice, A Bederson's five-point scale was used as the evaluation standard. According to this scoring standard, the score of the mice in the PBS group was 3.2, the score of the mice in the yeast peptide group was 1.5, and the score of the yeast peptide-hydroxytyrosol complex group was 1.2. As seen from the scoring results, the neurological score of the MCAO mice injected with the yeast peptide-hydroxytyrosol complex dispersion was significantly lower.

Similarly, this example used the same experimental method as described above to conduct neurological scoring of the mice, calculation of the area of cerebral infarction in the mice, and neurological function scoring of the mice for the prepared “Mu-1 complex”, which was a yeast peptide-hydroxytyrosol complex. The results were basically consistent with those of the “Mu-3 complex”, so they would not be repeated here.

The aforementioned results indicated that the yeast peptide-hydroxytyrosol complex prepared in this example was a complex with a stroke therapeutic characteristic.

To further evaluate the therapeutic effect of the yeast peptide-hydroxytyrosol complex on stroke, animal experiments were also designed in this example.

Experimental animals: SPF grade C57BL/6 male mice, 8 weeks old, weighing 20-25 g.

### Experimental Design:

1. Establishment of tMCAO model of mice: After the mice were anesthetized, their heads and limbs were fixed in a supine position. Following disinfection of the neck with iodine, a 1 cm midline incision was made. The right common carotid artery, external carotid artery, and internal carotid artery were successively isolated with forceps. The proximal ends of the common and external carotid arteries were ligated, and the distal end of the internal carotid artery was clamped with an arterial clip. A suture was reserved at the distal end of the common carotid artery, and an inverted V-shaped notch was cut at the proximal end and arterial bifurcation to allow entering of a filament. The filament was inserted into the internal carotid artery via the incision, and the pre-tied suture was tightened without hindering the movement of the filament. The arterial clip was removed, and the filament was advanced to the bifurcation of the internal and external arteries and fixed. The incision was sutured and treated with iodine to prevent infection. After 2 h of MCA (middle cerebral artery) occlusion, the filament was retracted by 10 mm to achieve blood reperfusion for 72 h. The modeling of the mice in the sham-operation group underwent the same procedure, except no filament was inserted.
2. Grouping and administration: The mice were randomly divided into 4 groups, with 10 mice in each group. The respective reagents were injected through the tail vein, with an injection volume of 200 μL. Experimental group 1 received an injection of the yeast peptide-hydroxytyrosol complex YP@HT prepared by the “Mu-3” yeast peptide in this example (formulated with a PBS buffer, at a concentration of 0.2 mg/mL), experimental group 2 received an injection of a “Mu-3” yeast peptide YP prepared in this example (at a concentration of 0.2 mg/mL), and experimental group 3 was injected with hydroxytyrosol HT (at a concentration 0.2 mg/mL). The sham-operation group and the control group were each injected with an equal volume of a PBS buffer.

### Evaluation of Brain Tissue Oxidative Damage:

After 7 days of administration, the mice were anesthetized and then sacrificed via cervical dislocation. Subsequently, the brain tissue was quickly extracted, and the olfactory bulbs were removed and the residual blood was rinsed with normal saline. 0.5 g of the brain tissue was taken, and blotted dry with filter paper to remove the surface moisture, added with normal saline in a ratio of 1:9, and then homogenized by centrifuging. Then the supernatant was collected. The ROS (reactive oxygen species) content, SOD (superoxide dismutase) activity, GSH (glutathione) content, and MDA (malondialdehyde) content in the brain tissue homogenate were measured using kits (all produced by Beyotime Bio) according to the instructions.

### Result Analysis:

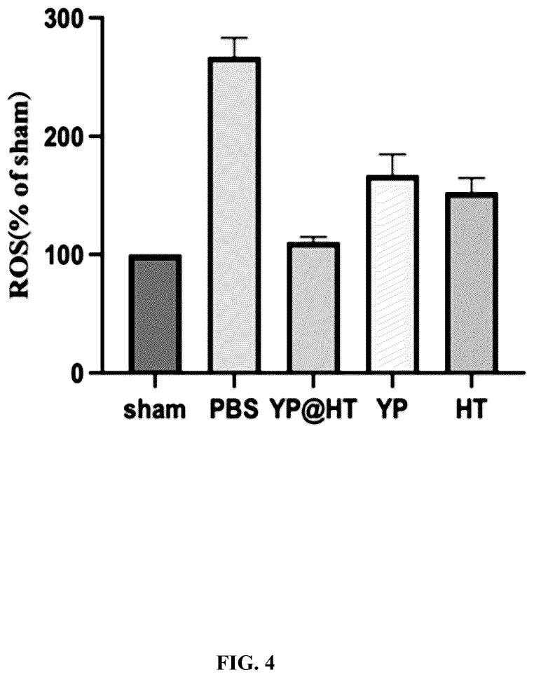

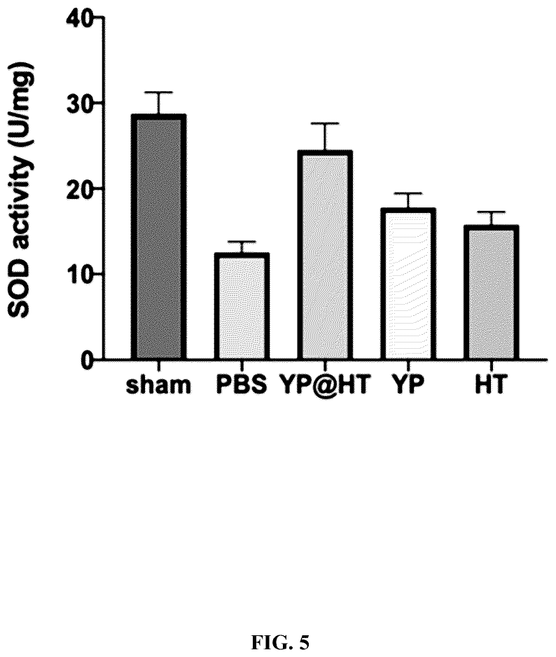

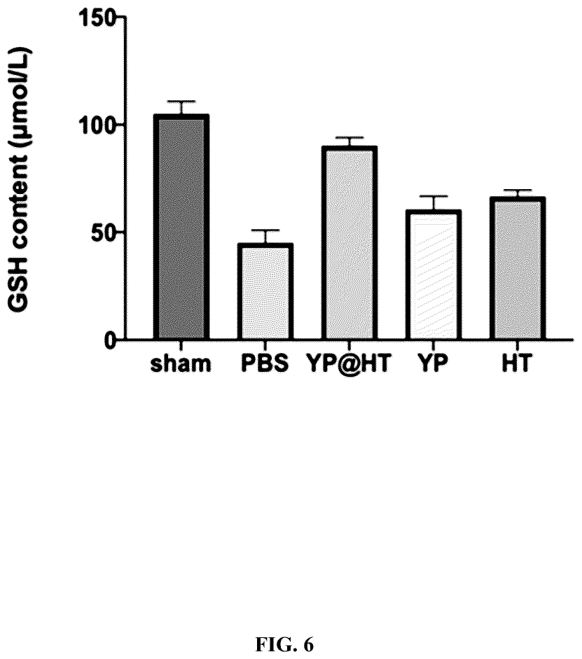

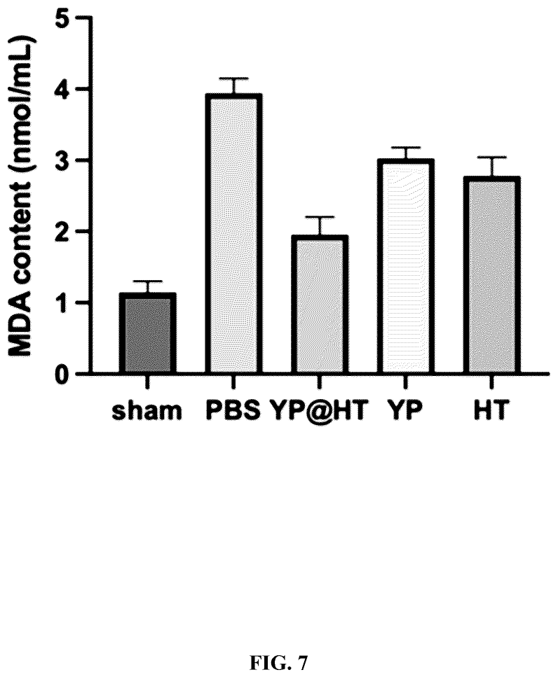

FIG. 4 showed the ROS detection results (the vertical axis showed that ROS content was multiple of the sham-operation group), FIG. 5 showed the detection results of SOD activity, FIG. 6 showed the detection results of GSH content, and FIG. 7 showed the detection results of MDA content. In FIGS. 4 to 7, “sham” on the horizontal axis represented the sham-operation group, “PBS” represented injection of a phosphate buffer, “YP@HT” represented injection of the “Mu-3 complex”, a yeast peptide-hydroxytyrosol complex prepared in this example, “YP” represented the Mu-3 yeast peptide prepared in this example, and “HT” represented hydroxytyrosol.

The occurrence and development of CIRI was closely associated with oxidative stress, with the surge in reactive oxygen species (ROSs) leading to oxidative stress. SOD was an endogenous antioxidant enzyme responsible for scavenging free radicals, while MDA was a lipid peroxidation product caused by oxidative stress (MDA levels were positively correlated with the extent of oxidative damage to cells). GSH could play a role in maintaining normal immune activity and had antioxidant activities. The aforementioned indicators could serve as biomarkers of the oxidative damage to nervous tissues.

As seen from FIGS. 4 to 7, compared with the Sham-operation group, the PBS group exhibited reduced levels of SOD and GSH, along with elevated MDA levels, indicating Oxidative damage in the brain tissues of the mice. The “MU-3 complex”, which was a yeast peptide-hydroxytyrosol complex, could effectively reverse the oxidative damage, with its effects superior to those of the yeast peptide and the hydroxytyrosol alone.

### Comparative Example 1

In this comparative example, a yeast peptide-hydroxytyrosol complex was prepared. The preparation method differed from that of the complex with a stroke therapeutic characteristic in the Example only in that during the preparation of the yeast peptide, alkaline protease (with a specification of 5.0×104 U/g) was used instead of papain, and the pH during enzymatic hydrolysis was adjusted to 9. Other operational methods and process parameters were the same as those in the Example.

### Comparative Example 2

In this comparative example, a yeast peptide-hydroxytyrosol complex was prepared. The preparation method differed from the preparation method of the complex with a stroke therapeutic characteristic in the Example only in that during the preparation of the yeast peptide, a flavor protease (with a specification of 3.0×104 U/mg) was used instead of papain. Other operation methods and process parameters were the same as those in the Example.

### Comparative Example 3

In this comparative example, a yeast peptide-hydroxytyrosol complex was prepared. The preparation method differed from the method for preparing the complex with a stroke therapeutic characteristic in the Example only in that during the preparation of the yeast peptide, bromelain (with a specification of 6.0×105 U/mg) was used instead of papain. Other operation methods and process parameters were the same as those in the Example.

### Comparative Example 4

In this comparative example, a yeast peptide-hydroxytyrosol complex was prepared. The preparation method differed from the preparation method of the complex with a stroke therapeutic characteristic in the Example only in that during the preparation of the yeast peptide, a commercially available compound protease (with a specification of 1.0×105 U/mg) without papain was used instead of papain. Other operation methods and process parameters were the same as those in the Example.

### Comparative Example 5

In this comparative example, a yeast peptide-hydroxytyrosol complex was prepared. The preparation method differed from the preparation method of the complex with a stroke therapeutic characteristic in the Example only in that during the preparation of the yeast peptide, the papain enzymatic hydrolysis temperature was set to 60° C., and the enzymatic hydrolysis time was 8 h. Other operation methods and process parameters were the same as those in the Example.

### Comparative Example 6

In this comparative example, a yeast peptide-hydroxytyrosol complex was prepared. The preparation method differed from the preparation method of the complex with a stroke therapeutic characteristic in the Example only in that during the preparation of the yeast peptide, an amount/mass ratio of the papain to the yeast protein in the enzymatic hydrolysis system was set to 12,000 U/g. Other operation methods and process parameters were the same as those in the Example.

When the yeast protein was enzymatically hydrolyzed to prepare the yeast peptides, the type of the enzyme, the amount of the enzyme, and the enzymatic hydrolysis conditions all influenced the size of the resulting yeast peptide fragments, which in turn affected the amino acid composition of the yeast peptides with molecular weights greater than or equal to 1 kDa and less than 3 kDa. Yeast peptides with different amino acid compositions exhibited distinct biological properties. To compare the efficacy of the yeast peptide-hydroxytyrosol complexes prepared in the Example and the Comparative Examples 1-6 in reducing oxidative stress damage to the neural tissue from CIRI (cerebral ischemia-reperfusion injury), the same method as described in the Example was used. The neural repair effects of the yeast peptide-hydroxytyrosol complexes from Comparative Examples 1-6 were respectively tested in the mouse tMCAO (transient middle cerebral artery occlusion) model.

Table 1 presented the measurement results for the reduction of oxidative stress damage in the brain tissues of tMCAO mice after injection of various yeast peptide-hydroxytyrosol complexes.

TABLE 1

| | | SOD | GSH | MDA |
|---|---|---|---|---|
| | ROS (% of | activity | content | content |
| Groups | sham) | (U/mg) | (μmol/L) | (nmol/mL) |
| Example | 110.5 | 24.5 | 90.25 | 1.95 |
| Comparative | 135.6 | 17.4 | 67.65 | 2.53 |
| Example 1 | | | | |
| Comparative | 137.3 | 18.0 | 64.23 | 2.49 |
| Example 2 | | | | |
| Comparative | 136.5 | 17.8 | 66.00 | 2.51 |
| Example 3 | | | | |
| Comparative | 138.0 | 18.2 | 65.00 | 2.50 |
| Example 4 | | | | |
| Comparative | 121.5 | 21.6 | 84.66 | 2.25 |
| Example 5 | | | | |
| Comparative | 116.7 | 22.7 | 88.39 | 2.08 |
| Example 6 | | | | |

From the results in the table, it can be seen that the mice injected with the “Mu-3 complex”, which is a yeast peptide-hydroxytyrosol complex prepared in the Example (the mice in the group of the Example) have a lower ROS content, a higher SOD activity, a higher GSH content, and a lower MDA content, indicating that the degree of oxidative stress damage to the nervous tissue of the mice in the group of the Example is lower. The yeast peptide-hydroxytyrosol complex prepared in the Example has a better effect in reducing oxidative stress damage to the nervous tissue of the mice in the tMCAO model compared to the yeast peptide-hydroxytyrosol complexes prepared in Comparative Examples 1 to 6.

Obviously, the above examples are merely examples provided for the purpose of clear explanation and are not intended to limit the implementations. Other different forms of changes or modifications can be made by those skilled in the art based on the above description. It is neither necessary nor possible to list all implementations here, and the obvious changes or modifications derived from these examples still fall within the scope of protection of the claims of this patent application.

What is claimed is:

1. A complex with a stroke therapeutic characteristic, comprising a yeast peptide and hydroxytyrosol, wherein the yeast peptide is a yeast peptide with a molecular weight less than 3 kDa produced by papain enzymolysis of a yeast protein; the pH during enzymatic hydrolysis is 6.8-7.2, the enzymatic hydrolysis time is 3.8-6.2 h, the enzymatic hydrolysis temperature is 48-58° C., and the amount/mass ratio of the papain to the yeast protein in the enzymatic hydrolysis system is 8,800-9,200 U/g.

2. The complex with a stroke therapeutic characteristic according to claim 1, further comprising lecithin and/or a phospholipid membrane prepared using lecithin, wherein the phospholipid membrane is prepared by dissolving the lecithin in chloroform and then evaporating the chloroform.

3. The complex with a stroke therapeutic characteristic according to claim 2, wherein when the lecithin is added into the complex: the molar ratio of the yeast peptide, the hydroxytyrosol and the lecithin is (6.5-38.9):(6.5-38.9):(0.1-0.5); and when the phospholipid membrane is added into the complex, the amount of the phospholipid membrane is calculated according to the amount of the lecithin used for preparing the phospholipid membrane: the molar ratio of the yeast peptide, the hydroxytyrosol and the phospholipid membrane is (6.5-38.9):(6.5-38.9):(0.1-0.5).

4. A method for preparing a complex with a stroke therapeutic characteristic, comprising the following steps:
(1) preparing yeast protein powder prepared from a yeast into a yeast protein powder dispersion;
(2) placing and shaking the yeast protein powder dispersion under an enzymatic hydrolysis reaction temperature;
(3) adding papain to the yeast protein powder dispersion for enzymatic hydrolysis, so as to obtaining an enzymatic hydrolyzate dispersion after the enzymatic hydrolysis is completed;
(4) heating the enzymatic hydrolyzate dispersion to a protease inactivation temperature and maintaining at this temperature, and then cooling the enzymatic hydrolyzate dispersion and adjusting the pH of the enzymatic hydrolyzate dispersion;
(5) centrifuging the enzymatic hydrolyzate dispersion and retaining the supernatant, and sequentially ultrafiltering and freeze-drying the supernatant to obtain a yeast peptide having a molecular weight less than 3 kDa;
(6) dispersing the yeast peptide and the hydroxytyrosol in a phosphate buffer to form a composite system, and stirring to obtain a yeast peptide-hydroxytyrosol complex dispersion;
(7) purifying and freeze-drying the yeast peptide-hydroxytyrosol complex dispersion to obtain a yeast peptide-hydroxytyrosol complex, wherein the yeast peptide-hydroxytyrosol complex is the complex with a stroke therapeutic characteristic according to claim 1.

5. The method for preparing a complex with a stroke therapeutic characteristic according to claim 4, wherein in step (1), a phosphate buffer is used for formulating a yeast protein powder dispersion, the pH of the phosphate buffer is equal to the pH during enzymatic hydrolysis; the content of the yeast protein in the yeast protein powder is greater than or equal to 80 wt %, and the concentration of the yeast protein powder in the yeast protein powder dispersion is 48-50 g/L.

6. The method for preparing a complex with a stroke therapeutic characteristic according to claim 4, wherein in step (2), the shaking time is 10-15 min; in step (4), the protease inactivation temperature is 95-100° C., and the heat preservation time is 10-15 minutes; after the enzymatic hydrolysate is naturally cooled, the pH of the enzymatic hydrolysate is adjusted to 7.0.

7. The method for preparing a complex with a stroke therapeutic characteristic according to claim 4, wherein in step (5), during centrifugation of the enzymatic hydrolysate, the centrifugation temperature is 4-8° C., the rotation speed during centrifugation is 10,000-12,000 rpm, and the centrifugation time is 10-20 min;
in step (6), the molar ratio of the yeast peptide to the hydroxytyrosol in the composite system is 1:1, and the concentration of the hydroxytyrosol in the composite system is 1-6 mg/mL; after the composite system is prepared, lecithin and/or a phospholipid membrane made of lecithin is added into the composite system; the molar amount of the phospholipid membrane in the composite system is calculated according to the molar amount of the lecithin, and the molar concentration of the lecithin in the composite system is 0.1-0.5 mmol/L.

8. The method for preparing a complex with a stroke therapeutic characteristic according to claim 4, wherein in step (6), the composite system is subjected to cyclic heating during the stirring process; the cyclic heating method is to heat the composite system to 48-52° C. at a heating rate of 3-7° C./min, maintaining at this temperature for 20-30 min, and then naturally cooling to room temperature until the stirring is completed; wherein the stirring time is 20-26 h.

9. The method for preparing a complex with stroke therapeutic characteristic according to claim 4, wherein in step (1), a yeast protein powder dispersion is prepared using a phosphate buffer, the pH of the phosphate buffer is 7.0; the content of the yeast protein in the yeast protein powder is greater than or equal to 80 wt %, and the concentration of the yeast protein powder in the yeast protein powder dispersion is 50 g/L;
in step (2), the shaking time is 10 min;
in step (3), the enzymatic hydrolysis time is 6 h, the enzymatic hydrolysis temperature is 55° C., the pH during enzymatic hydrolysis is 7, and the ratio of the papain to the yeast protein in the enzymatic hydrolysis system is 9,000 U/g;
in step (4), the protease inactivation temperature is 100° C., and the heat preservation time is 15 min; after the enzymatic hydrolyzate dispersion is naturally cooled, the pH of the enzymatic hydrolyzate dispersion is adjusted to 7.0;
in step (5), during centrifugation of the enzymatic hydrolyzate dispersion, the centrifugation temperature is 4° C., the centrifugation speed is 10,000 rpm, and the centrifugation time is 15 min;
in step (6), the molar ratio of the yeast peptide to the hydroxytyrosol in the composite system is 1:1, and the concentration of the hydroxytyrosol in the composite system is 5 mg/mL; after the composite system is prepared, lecithin and/or a phospholipid membrane made of lecithin is added into the composite system; the amount of the phospholipid membrane in the composite system is calculated according to the molar concentration of the lecithin, and the molar concentration of the lecithin in the composite system is 0.1 mmol/L; during the stirring process, the composite system is subjected to cyclic heating; the method of the cyclic heating is to heat the composite system to 50° C. at a heating rate of 5° C./min, maintaining at this temperature for 30 min, and then naturally cooling to room temperature until the stirring ends; and the stirring time is 24 h.

10. Use of the complex with a stroke therapeutic characteristic according to claim 1 in manufacture of a drug for treating ischemic stroke.
